(19) United States
(12) Patent Application Publication
(10) Pub. No.: US 2006/0149136 A1
(43) Pub. Date: Jul. 6, 2006

(54) Elongating balloon device and method for soft tissue expansion

(75) Inventors: Christine Seto, Palo Alto, CA (US); Nenita Salom, San Jose, CA (US); Cesar Ico, San Francisco, CA (US); Richard W. Layne, Sunnyvale, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Kyphon Inc., Sunnyvale, CA (US)

(21) Appl. No.: 11/021,786

(22) Filed: Dec. 22, 2004

(51) Int. Cl. A61B 1/32

(52) U.S. Cl. 600/204

(57) ABSTRACT

Tissue distractor devices, systems, and methods for soft tissue expansion in order to create a space between the tissue to improve visualization and for increased working space during surgery are disclosed. One method for separating soft tissues for cannula access to a treatment site within a patient body comprises providing a catheter having a tissue distractor on a distal end thereof and positioning the catheter between the soft tissues near the treatment site. Once properly positioned, the tissue distractor is expanded so as to separate the soft tissues. The tissue distractor is then elongated by mechanical actuation so as to provide cannula access to the treatment site. The cannula forms a working channel for surgical procedures, particularly minimally invasive surgical procedures.

### BACKGROUND OF THE INVENTION

1. Field of the Invention

The present invention relates generally to tissue distractors for soft tissue expansion in the medical field. More particularly, the present invention relates to elongating balloon devices, systems, and methods for separating soft tissues apart for cannula access to a treatment site within a patient body.

Generally, tissue distractors are used for expanding or separating tissues in order to create a space between the tissue to improve visualization and for increased working space during open surgery and minimally invasive surgery. Current methods used for increasing work space and improving visualization employ mechanical separators, such as metal retractors, scalpels, trocars, etc. Conventional metal retractors are often bulky and awkward and require substantially large open incisions in a skin surface which may damage large amounts of healthy tissue. Metal retractors often tear the muscle and tissue fibers apart, thereby increasing tissue trauma and healing time. Further, excess pressure from metal retractors may cause necrosis or tissue death, as it is difficult to monitor the pressure being applied to the body tissues. Other methods for separating tissue employ direct pressure of an unconfined flow of fluid such as water or saline during surgery. Problems with water pressurization include fluid extravasation including into and through the tissue itself. Further, increased pressure and swelling may result in the area, which may lead to edematous or swollen tissue. Excess water pressure may also lead to tissue damage or necrosis, as it difficult to monitor the pressure being applied to the body tissues.

Another class of devices commercially and medically in use are fluid operated retractors, such as those issued to Bonutti and described in U.S. Pat. No. 5,331,975, licensed to the assignee of the present application. Fluid operated retractors, such as an inflatable balloon or bladder, allow a surgeon to take potential spaces within the body and turn them into existing spaces safely, easily, and controllably in order to safely visualize appropriate tissue and operate. Such devices allow for selective retraction of tissue, either of hard tissue such as bone or soft tissue planes, to be moved out of the way to improve working space and visualization, which is of particular benefit while operating from within the body, e.g., minimally invasive surgery. These tissue retractors also permit working within the body without damaging a great deal of tissue in the path between a skin opening and the working area, by minimizing the external orifice or skin incision. Although such devices have achieved relative levels of success, improvements to such balloon tissue retraction devices would be advantageous. For example, such devices may be modified to assist in cannula access to a treatment site within a patient body for surgery.

2. Description of the Background Art

Devices and methods for expanding or separating soft tissues are described in U.S. Pat. Nos. 5,163,949; 5,197,971; 5,295,994; 5,331,975; 5,345,927; 5,514,153; 5,601,590; 5,667,520; 5,685,826; 5,707,390; 5,716,325; 5,827,318; 5,860,997; 5,888,196; 5,954,739; 6,017,305; 6,042,596; 6,102,928; 6,171,236; 6,171,299; 6,187,023; 6,277,136; 6,358,266; 6,451,042; and 6,620,181, all of which are licensed to the assignee of the present application. A paraspinal approach between layers of tissue to the spine is described in an article by Wiltse et al., New Uses and Refinements of the Paraspinal Approach to the Lumbar Spine, Spine 13(6), 696-706 (1998). U.S. Pat. No. 5,795,325 describes an endoaortic catheter having an occluding member for temporarily inducing cardioplegic arrest in the heart of a patient and for establishing cardiopulmonary bypass in order to facilitate heart and blood vessel surgical procedures. U.S. Pat. No. 6,352,501 describes a radiation source for intravascular application of radiation to treat vascular disease. U.S. Pat. No. 6,537,247 describes an intravascular dilation catheter having a funnel shaped shroud tube for clearing blocked body lumens.

The full disclosures of each of the above mentioned references are incorporated herein by reference in their entirety.

### BRIEF SUMMARY OF THE INVENTION

The present invention provides tissue distractor devices, systems, and methods for soft tissue expansion in order to create a space between the tissue to improve visualization and for increased working space during open surgery and minimally invasive surgery. In particular, the present invention provides elongating balloon devices that are mechanically actuated for separating soft tissues apart for cannula access to a treatment site within a patient body. The cannula forms a working channel for surgical procedures, particularly minimally invasive surgical procedures. For example, the cannula may provide access through spinal tissue to the spine for inserting spinal instrumentation, such as screws, rods, plates, disc, etc., for treating vertebrae factures. It will be appreciated however that the present invention is not limited by the example of minimally invasive spinal surgery, and as such may be applied to open surgical procedures as well as for distracting other tissue parts in the body, such as tissues of the arms, legs, face, or vertebral discs for orthopedic or cosmetic surgery. Further, the term “soft tissue” used herein includes tissue planes, adjacent layers of tissue, muscle and tissue fibers.

In a first aspect of the present invention, a method for separating soft tissues, such as spinal tissues, for cannula access to a treatment site, such as a spine, within a patient body is provided. The method comprises providing a catheter having a tissue distractor on a distal end thereof and positioning the catheter between the soft tissues near the treatment site from a side, anterior, or preferably a posterior approach. Once properly positioned, the tissue distractor is expanded so as to separate the soft tissues. The tissue distractor is then elongated or stretched by mechanical actuation so as to provide cannula access to the treatment site.

The tissue distractor, preferably a balloon, bladder, or other expansible element capable of creating a void, is expanded by inflating the balloon with fluid under pressure. Inflating generally comprises radial dilation of the balloon so as to gently spread apart the soft tissues radially outward. It will be appreciated that balloon expansion gently pulls the muscle and tissue fibers apart with minimal complications, such as swollen tissue, patient trauma, cutting or tearing, and healing delays. The balloon may be controllably inflated to a volume in a range from about 3 cc to about 30 cc, preferably in range from about 10 cc to about 18 cc so as to safely and easily create an existing space. The balloon volume will remain substantially constant prior to and subsequent to balloon elongation. The balloon may be inflated to a diameter (e.g., over-dilated), prior to elongation, that is larger than a diameter of the cannula so as to effectively expand the tissue prior to cannula advancement.

Balloon elongation may be carried out by a variety of mechanical actuation means. For example, balloon elongation may be performed by pushing distally on an inner shaft of the catheter relative to an outer shaft of the catheter, wherein a proximal end of the balloon is coupled to the outer shaft and a distal end of the balloon in coupled to the inner shaft. Optionally, balloon elongation may be performed by pulling proximally on the outer shaft of the catheter relative to the inner shaft of the catheter, wherein a proximal end of the balloon is coupled to the outer shaft and a distal end of the balloon in coupled to the inner shaft. Still further, the outer and inner shafts may be rotatably configured with respect to one another to allow for balloon elongation. The outer and inner shaft may be formed from rigid materials to further aid in balloon penetration between the soft tissues.

The balloon is elongated in an axial direction, typically to an elongated length in a range from about 60 mm to about 180 mm, preferably in a range from about 80 mm to about 100 mm. Balloon elongation reduces an outer diameter of the balloon so that the cannula may be easily advanced over the elongated balloon without issues of interference between the balloon and/or balloon and soft tissue interface. Further, balloon elongation does not require balloon deflation for cannula positioning, which process is often cumbersome and may lead to tissue retraction. Rather, in the present invention balloon volume during elongation remains substantially constant. Balloon elongation and cannula advancement may be carried out simultaneously, which is of preference, or sequentially. This process of inflation and elongation may be repeated until the desired depth is reached. For example, the balloon may be moved along the surgical site once or several times until the cannula is positioned within the soft tissues near the treatment site. The balloon catheter may then be deflated and retracted, leaving the cannula behind to form the working channel.

The method of the present invention further comprises making a small, shallow incision in a skin surface prior to positioning the catheter. As discussed above, balloon tissue distractors advantageously permit working within the body without damaging a great deal of tissue in the path between a skin opening and the working area, by minimizing the external orifice or skin incision. Such a small incision may be made with a scalpel, stylet, trocar, or the like. Alternatively, the balloon may be equipped with a cutting tip. In either protocol, positioning the catheter comprises inserting the catheter through the small incision in the skin and between the soft tissues. The tissue distractor may further be imaged, for example under fluoroscopy, so that a physician may view and aid in proper positioning, expansion, and elongation of the balloon tissue distractor.

In another aspect of the present invention, a method for separating spinal tissues for cannula access to a spine within a patient body for performing minimally invasive spinal surgery is provided. The method comprises providing a catheter having a balloon on a distal end thereof. The balloon catheter is positioned between spinal tissues near the spine. The balloon is then inflated so as to expand and spread apart the spinal tissues. The balloon is then elongated by mechanical actuation to allow for cannula advancement and positioning near the spine.

In a further aspect of the present invention, a device for separating soft tissues for cannula access to a treatment site within a patient body is provided. The device comprises a catheter body having a proximal end and a distal end. An expansible tissue distractor is disposed on the distal end of the catheter. A mechanical actuation mechanism is coupled to the tissue distractor for elongating the tissue distractor. The tissue distractor preferably comprises a balloon formed from non-compliant, semi-compliant, or compliant materials. The balloon may be formed from a variety of medical grade materials including TEXIN®, polyurethane, polyethylene, polyethylene terephthalate, polytetrafluroethylene, nylon, silicone, latex, polyvinyl chloride, thermoplastic elastomer, elastic materials, radiopaque materials and combinations thereof. Exemplary balloon materials and extrusion processes are described in greater detail in U.S. Pat. No. 6,607,544, which is assigned to the assignee of the present application and incorporated herein by reference.

The device may further comprise an inflation assembly in fluid communication with the balloon so as to inflate the balloon, prior to elongation, to an expanded diameter. The balloon may be inflatable with a variety of media including air, fluid (e.g., water, saline, dextrose water, CO₂, N₂), radiopaque medium, silicone, gels, solid materials (e.g., pellets), and combinations thereof. For example, a radiopaque filled balloon further allows the distractor to be imaged, for example under fluoroscopy, so that a physician may view and aid in proper positioning, expansion, and elongation of the balloon tissue distractor. Preferably, the balloon is filled with safe fluid, such as water or saline, in the event of accidental fluid leakage into the body.

The tissue distractor is initially placed into the body in an unexpanded configuration and then as it is inflated into an expanded configuration, tissue is pushed out of the way into deeper layers of the body to improve visualization and operation at a surgical site. The expanded balloon is flexible and thus there are no sharp edges which might injure tissue being moved by the distractor. Further, the expanded balloon conforms to the tissue confines and the exact pressure can be monitored and regulated to keep the force exerted by the distractor at a safe level to minimize any tissue damage. The expanded balloon diameter, prior to elongation, may be in range from about 5 mm to about 50 mm, preferably in range from about 15 mm to about 25 mm. The expanded balloon diameter, prior to elongation, may be larger than a diameter of the cannula so as to effectively expand the tissue prior to cannula advancement. The expanded balloon may have a volume in a range from about 3 cc to about 30 cc, preferably in range from about 10 cc to about 18 cc. The balloon volume may be substantially maintained prior to and subsequent to balloon elongation.

The mechanical actuation mechanism may comprise an inner shaft of the catheter that is distally moveable relative to an outer shaft of the catheter. In this embodiment, a plunger may additionally be coupled to the proximal end of the inner shaft to elongate the balloon tissue distractor. In another embodiment, the mechanical actuation mechanism may comprise an outer shaft of the catheter that is proximally moveable relative to an inner shaft of the catheter. In either embodiment, a proximal end of the balloon is coupled to a distal end of the outer shaft and a distal end of the balloon is coupled to a distal end of the inner shaft.

Preferably, the inner and/or outer shaft is rigid so that the balloon may be effectively penetrated into the tissue and elongated. The inner and/or outer shaft may comprise a hypotube formed from a variety of medical grade materials including metal (e.g., stainless steel, NITINOL®), plastic, polymer (e.g., TEXIN®, polyurethane), and combinations thereof. The hypotube may alternatively comprise a separate tubing disposed over the shaft or still further an outer jacket may be disposed over the shaft and/or hypotube, the jacket and hypotube being formed from similar medical grade materials as those described above with respect to the shafts. An inflation lumen may additionally be defined between or within the inner and/or outer shafts for fluid communication between the inflation assembly and the balloon tissue distractor.

The balloon tissue distractor has a variable or adjustable length in a range from about 40 mm to about 150 mm, preferably in range from about 75 mm to about 95 mm. The balloon is then elongated in an axial direction, typically to an elongated length in a range from about 60 mm to about 180 mm, preferably in a range from about 80 mm to about 100 mm. Balloon elongation reduces an outer diameter of the balloon so that the elongated balloon diameter is smaller than a diameter of the cannula. In this way, the cannula may be easily shuttled over the elongated balloon without issues of interference between the balloon and/or balloon and soft tissue interface. Typically, the change in the outer diameter of the elongated balloon is reduced to a range from about 0.1 mm to about 10 mm, preferably from about 0.5 mm to about 3 mm.

In yet another aspect of the present invention, a system for separating spinal tissues for cannula access to a spine within a patient body is provided. The system comprises a catheter body and a cannula disposed over the catheter body. The catheter body has a balloon tissue distractor disposed on a distal end thereof configured to separate spinal tissues near the spine and a mechanically actuatable elongation mechanism coupled to the balloon tissue distractor. The balloon tissue distractor and mechanical actuation mechanism for balloon elongation have already been described herein. The working cannula may be formed from a variety of medical grade tubing including metal (e.g., stainless steel, NITINOL®), plastic, polymer (e.g., PEBAX®), and combinations thereof. The cannula will generally have a diameter in a range from about 0.5 inch to about 1.5 inches, preferably from about 0.7 inch to about 1 inch. The cannula may be a separate component of the system or alternatively form a partially integrated system with balloon catheter.

A further understanding of the nature and advantages of the present invention will become apparent by reference to the remaining portions of the specification and drawings.

### BRIEF DESCRIPTION OF THE DRAWINGS

The following drawings should be read with reference to the detailed description. Like numbers in different drawings refer to like elements. The drawings, which are not necessarily to scale, illustratively depict embodiments of the present invention and are not intended to limit the scope of the invention.

FIGS. 1A through 1D illustrate side and cross sectional views an exemplary elongating tissue distractor device constructed in accordance with the principles of the present invention.

FIGS. 2A through 2D illustrate side and cross sectional views of another embodiment of the elongating tissue distractor device constructed in accordance with the principles of the present invention.

FIGS. 3A and 3B illustrate cross sectional views of a system including an elongating tissue distractor device and a cannula, wherein the tissue distractor is shown in an expanded configuration and an expanded, elongated configuration respectively.

FIGS. 4A and 4B illustrate a method for separating soft tissues for cannula access to a treatment site within a patient body employing the elongating tissue distractor device of FIG. 1A.

### DETAILED DESCRIPTION OF THE INVENTION

The present invention provides elongating balloon tissue distractors that are mechanically actuated for separating soft tissues apart for cannula access to a treatment site within a patient body. The cannula forms a working channel for surgical procedures, particularly minimally invasive surgical procedures. For example, the cannula may provide access through spinal tissue to the spine for inserting spinal instrumentation, such as screws, rods, plates, disc, etc., for treating vertebrae factures. It will be appreciated however that the present invention is not limited by the example of minimally invasive spinal surgery, and as such may be applied to open surgical procedures as well as for distracting other tissue parts in the body for access to various treatment sites. Further, the term “soft tissue” used herein includes tissue planes, adjacent layers of tissue, muscle and tissue fibers. The present invention is not however limited to soft tissue expansion, and as such may be further utilized to expand hard tissues, such as bones, joints, or other body parts.

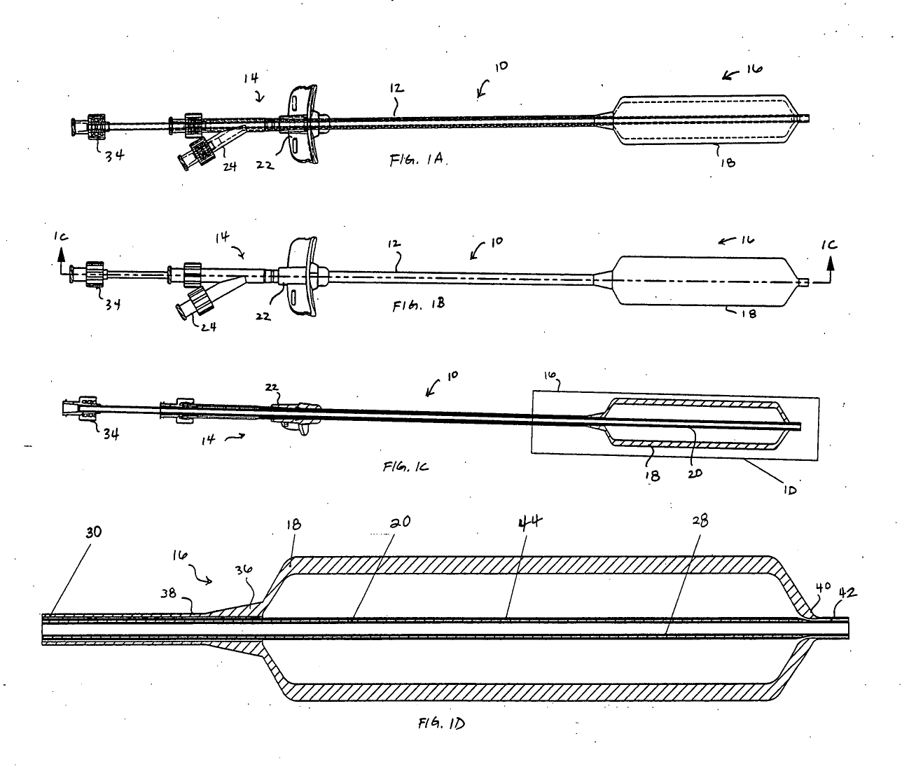

Referring to FIGS. 1A through 1D, side and cross sectional views of an exemplary elongating tissue distractor device 10 constructed in accordance with the principles of the present invention are illustrated. The device 10 comprises a catheter body 12 having a proximal end 14 and a distal end 16, as shown in FIGS. 1A and 1B. An expansible tissue distractor 18 is disposed on the distal end of the catheter. A mechanical actuation mechanism 20 is coupled to the tissue distractor 18 for elongating the tissue distractor 18, as best seen in the cross sectional views of FIGS. 1C and 1D. A hub 22, such as a Y connector or Luer cap, may be attached to the proximal end 14 of the catheter body 12 as will be discussed in more detail below. It will be appreciated that the above depictions are for illustrative purposes only and do not necessarily reflect the actual shape, size, or dimensions of the device 10. This applies to all depictions hereinafter.

The expansible tissue distractor 18 comprises a balloon formed from non-compliant, semi-compliant, or compliant materials. Alternatively, the tissue distractor may comprise a bladder or other expansible structures that are capable of creating a void. The balloon tissue distractor 18 may be formed from a variety of medical grade materials including TEXIN®, polyurethane, polyethylene, polyethylene terephthalate, polytetrafluroethylene, nylon, silicone, latex, polyvinyl chloride, thermoplastic elastomer, elastic materials, and combinations thereof. Preferably, the balloon 18 comprises a compliant material, such as TEXIN®. In general, the balloon tissue distractor 18 should be capable of interfacing with the cannula or other tools so that balloon performance is optimized. An inflation assembly (not shown) may be attached to an end 24 of the hub 22 so as to be in fluid communication with the balloon tissue distractor 18 so as to inflate the balloon 18, prior to elongation, to an expanded diameter. The balloon tissue distractor 18 may be inflatable with a variety of media including air, fluid (e.g., water, saline, dextrose water, C0₂, N₂), radiopaque medium, silicone, gels, solid materials, and combinations thereof.

The balloon tissue distractor 18 is initially placed into a patient body in an unexpanded configuration and then as it is inflated into an expanded configuration, tissue is gently pushed out of the way as the balloon tissue distractor is inserted into deeper layers of the body to improve visualization and operation at a surgical site. The expanded balloon 18 is flexible and thus there are no sharp edges which might injure tissue being moved by the distractor. Further, the fluid filled balloon 18 conforms to the tissue confines and the exact pressure can be monitored and regulated to keep the force exerted by the distractor 18 at a safe level to minimize any tissue damage.

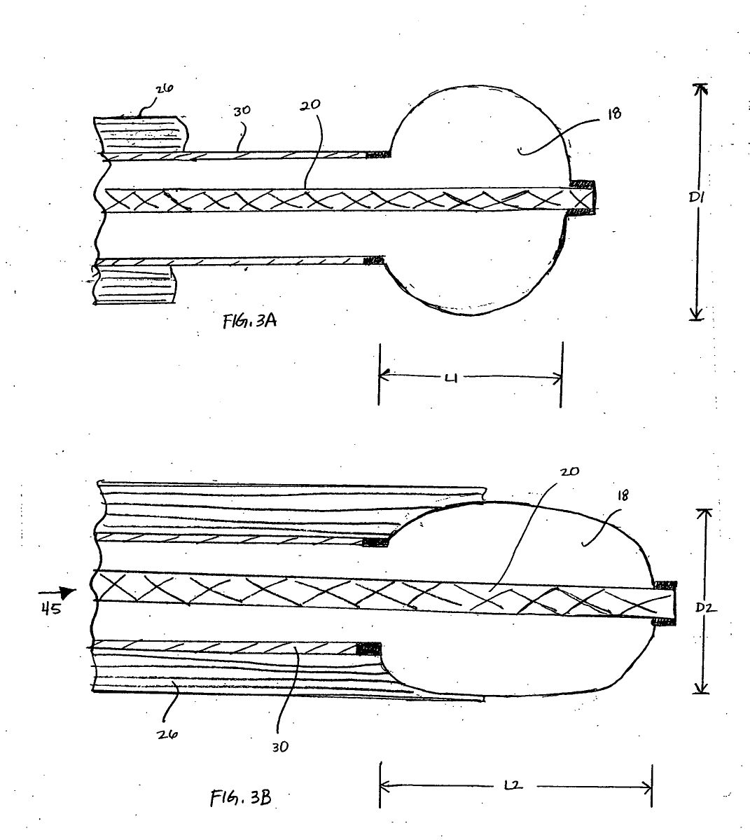

The balloon tissue distractor 18 is shown in an expanded configuration in FIG. 3A, wherein the expanded balloon diameter D1, prior to elongation, may be in range from about 5 mm to about 50 mm, preferably in range from about 15 mm to about 25 mm. The expanded balloon diameter D1, prior to elongation, may be larger than a diameter of the cannula 26 so as to effectively expand the tissue prior to cannula advancement. The expanded balloon 18 may have a volume in a range from about 3 ccc to about 30 cc, preferably in range from about 10 cc to about 18 cc. The balloon volume may be substantially maintained prior to and subsequent to balloon elongation. The balloon tissue distractor 18 will have a variable or adjustable length L1, prior to elongation, in a range from about 40 mm to about 150 mm, preferably in range from about 75 mm to about 95 mm.

Referring back to FIG. 1D, the catheter body 12 has a coaxial design of concentric shafts 28 and 30. In this embodiment, the mechanical actuation mechanism 20 comprises a hypotube 20 disposed over an inner shaft 28 of the catheter body 12 that is distally moveable relative to an outer shaft 30 of the catheter body 12. A proximal end 36 of the balloon 18 is bonded to a distal end 38 of the outer shaft 30 and a distal end 40 of the balloon 18 is bonded to a distal end 42 of the inner shaft 28 and/or hypotube 20 with the use of adhesives and like fasteners. In this embodiment, a plunger 32 may additionally be coupled to or integrally formed with the proximal end of the hypotube 20 and extend outwardly from another end 34 of the hub 22 on the proximal end 14 of the catheter body 12 to elongate the balloon tissue distractor 28.

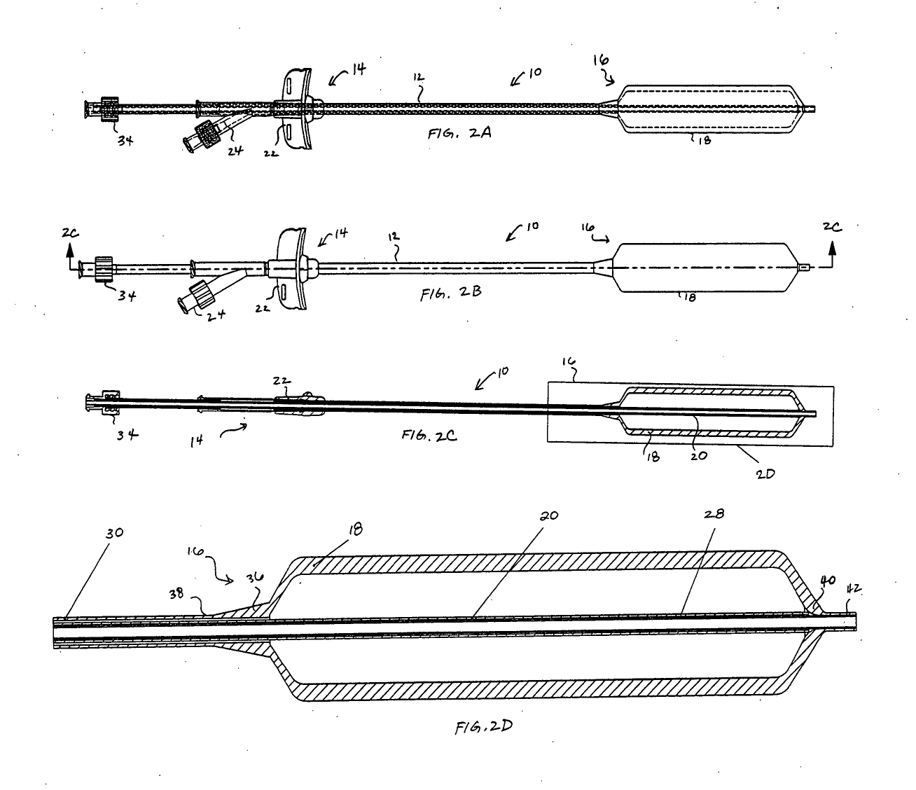

The embodiment of FIGS. 2A through 2D is similar in all respects to the embodiment of FIGS. 1A though 1D, with the exception that a plastic outer jacket 44 is disposed over the hypotube 20 in the latter embodiment, as best seen in FIG. 1D. Preferably, the hypotube 20 is rigid so that the balloon may be effectively penetrated into the tissue and elongated. The hypotube 20, inner shaft 28, outer shaft 30, hub 22, and plunger 32 may be formed from a variety of medical grade materials including metal (e.g., stainless steel, NITINOL®), plastic, polymer (e.g., TEXIN®, polyurethane), and combinations thereof. An inflation lumen may additionally be defined between or within the inner and/or outer shafts 28 and 30 for fluid communication between the inflation assembly and the balloon tissue distractor 18.

The balloon tissue distractor 18 is shown in an expanded, elongated configuration in FIG. 3B. The balloon 18 is elongated in an axial direction by pushing distally on the hypotube 20 and/or inner sheath 28, as depicted by arrow 45. Typically the balloon 18 has an elongated length L2 in a range from about 60 mm to about 180 mm, preferably in a range from about 80 mm to about 100 mm. Balloon elongation reduces an outer diameter of the balloon 18 so that the elongated balloon diameter D2 is smaller than a diameter of the cannula 26. In this way, the cannula 26 may be easily shuttled over the elongated balloon without issues of interference between the balloon and/or balloon and soft tissue interface. Typically, the change in the elongated balloon diameter D2 is reduced by a range from about 0.1 mm to about 10 mm, preferably from about 0.5 mm to about 3 mm.

The present invention advantageously reduces the diameter of the expanded balloon sufficiently so that the cannula can easily slide or shuttle over the elongated balloon after the balloon has gently spread the tissue apart. The dimensions described herein are for a typical balloon used to distract tissues along, near, or within the spine using a posterior approach. It will be appreciated however that the balloon may take on a variety of dimensions. For example, the length, diameter, and volume of the balloon may vary depending on the depth of penetration into tissue and the width of the access opening to the surgical site. For instance, the balloon may have longer dimensions if an approach from the anterior or lateral side is taken.

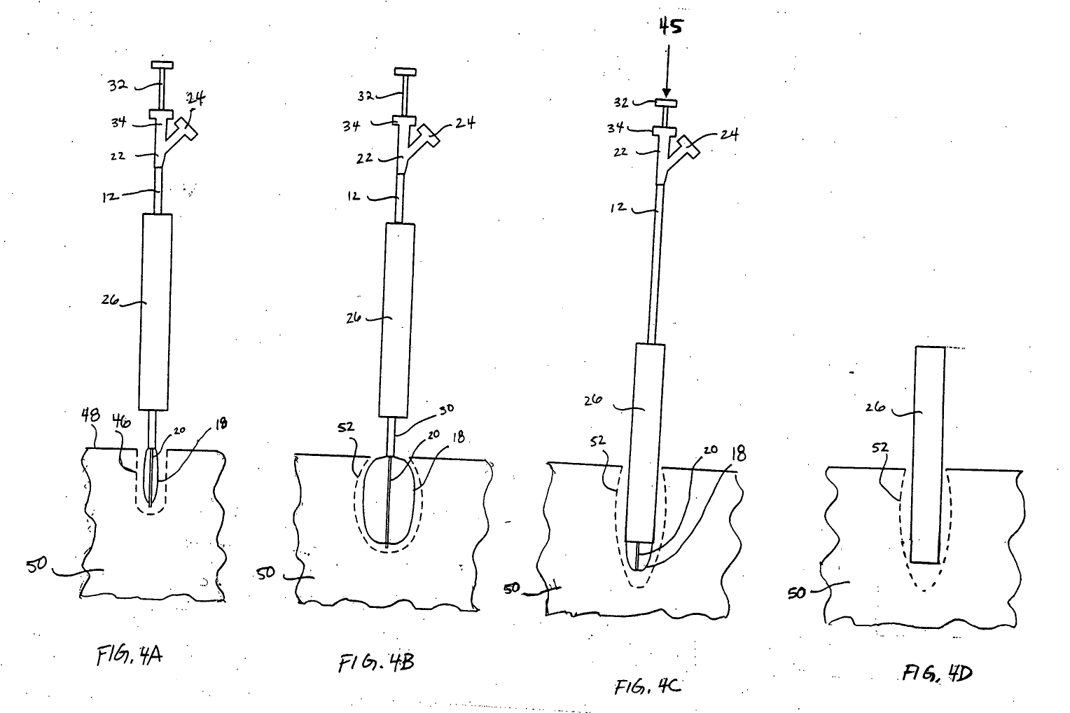

Referring now to FIGS. 4A through 4D, a method illustrating how the elongating balloon device 10 of the present invention assists in cannula 26 positioning into soft spinal tissues for access to a spine within a patient body is illustrated. The method comprises providing a catheter 12 having a balloon tissue distractor 18 on a distal end 16 thereof and a working cannula 26. A small, shallow incision 46 is initially made in a skin surface 48 prior to positioning the catheter 12. As discussed above, the balloon tissue distractor 18 advantageously permits working within the body without damaging a great deal of tissue in the path between a skin opening and the working area, by minimizing the external orifice or skin incision. Such a small incision 46 may be made with a scalpel, stylet, trocar, or the like. As shown in FIG. 4A, the catheter 12 is then inserted through the small incision 46 in the skin 48 and between the soft tissues 50. Typically, the catheter 12 is positioned from a posterior approach, e.g. from the patient's back and between the tissue 50 near the spine as such an approach results in less cutting and reduced mortality rates. The tissue distractor 18 may further be imaged, for example under fluoroscopy, so that a physician may view and aid in proper positioning, expansion, and elongation of the balloon tissue distractor.

Once properly positioned, the balloon tissue distractor 18 is expanded so as to separate the soft tissues 50 as best seen in FIG. 4B. The balloon tissue distractor 18 creates a void 52 by inflation of the balloon with fluid under pressure. Balloon inflation may be carried out manually or via automatic operation. Inflating generally comprises radial dilation (D1 in FIG. 3A) of the balloon 18 so as to gently spread apart the soft tissues 50 radially outward. It will be appreciated that balloon expansion gently pulls the muscle and tissue fibers apart with minimal complications, such as trauma to surrounding tissues and healing delays. The balloon 18 may be controllably inflated to a desired volume so as to safely and easily create an existing space 52. The balloon volume will remain substantially constant prior to and subsequent to balloon elongation. The balloon 18 is inflated to a diameter D1, prior to elongation, that is larger than a diameter of the cannula 26 so as to ensure adequate tissue expansion 52 prior to cannula 26 advancement.

As shown in FIG. 4C, the tissue distractor 18 is then elongated or stretched by mechanical actuation, as denoted by arrow 45, so as to provide cannula 26 access to the treatment site. Balloon elongation may be performed by pushing distally on the proximal plunger 32 so as to move the hypotube 20 and/or inner shaft 28 of the catheter 12 relative to an outer shaft 30 of the catheter 12. The hypotube 20 and/or inner shaft 28 may be formed from rigid materials, such as metal, to further aid in catheter 12 positioning between the soft tissues 50 whether the balloon 18 is deflated (FIG. 4A) or inflated (FIGS. 4B and 4C). The balloon 18 is elongated in an axial direction to an elongated length L2. Balloon elongation reduces an outer diameter D2 of the balloon so that the cannula 26 may be easily advanced over the elongated balloon 18 without issues of interference between the balloon 18 and/or balloon 18 and soft tissue interface 50.

Advantageously, balloon elongation does not require balloon deflation which in turn simplifies the cannula positioning procedure as well as prevents undesirable tissue retraction. Balloon elongation and cannula advancement may be carried out manually or via automatic operation, wherein such protocols may be combined to make the procedure simpler and faster. This process of inflation (FIG. 4B) and elongation (FIG. 4C) may be repeated until the desired depth is reached. For example, the balloon may be moved along the surgical site once if a longer balloon is utilized. Alternatively, the balloon may be shuttled several times if there is substantial spinal tissue 50 separating the balloon 18 from the spine. As shown in FIG. 4D, the balloon catheter may then be deflated and retracted, leaving the cannula 26 behind to form the working channel.

Although certain exemplary embodiments and methods have been described in some detail, for clarity of understanding and by way of example, it will be apparent from the foregoing disclosure to those skilled in the art that variations, modifications, changes, and adaptations of such embodiments and methods may be made without departing from the true spirit and scope of the invention. Therefore, the above description should not be taken as limiting the scope of the invention which is defined by the appended claims.

What is claimed is:

1. A method for separating soft tissues for cannula access to a treatment site within a patient body, the method comprising:
providing a catheter having a tissue distractor on a distal end thereof;
positioning the catheter between soft tissues near the treatment site;
expanding the tissue distractor so as to separate the soft tissues; and
elongating the tissue distractor by mechanical actuation.

2. The method of claim 1, wherein the soft tissues comprise spinal tissues and the treatment site comprises a spine.

3. A method as in claim 1, wherein the tissue distractor comprises a balloon and expanding comprises inflating the balloon.

4. The method of claim 3, wherein inflating comprises radial dilation of the balloon so as to spread apart the soft tissues radially outward.

5. The method of claim 3, wherein the balloon is inflated to a volume in a range from about 3 cc to about 30 cc.

6. The method of claim 5, wherein the balloon volume remains substantially constant prior to and subsequent to balloon elongation.

7. The method of claim 3, wherein the balloon has an inflated diameter, prior to elongation, that is larger than a diameter of the cannula.

8. The method of claim 3, wherein balloon elongation is performed by pushing distally on an inner shaft of the catheter relative to an outer shaft of the catheter, wherein a proximal end of the balloon is coupled to the outer shaft and a distal end of the balloon is coupled to the inner shaft.

9. The method of claim 3, wherein balloon elongation is performed by pulling proximally on an outer shaft of the catheter relative to an inner shaft of the catheter, wherein a proximal end of the balloon is coupled to the outer shaft and a distal end of the balloon is coupled to the inner shaft.

10. The method of claim 3, wherein balloon elongation reduces an outer diameter of the balloon.

11. The method of claim 3, further comprising advancing the cannula over the elongated balloon.

12. The method of claim 11, wherein elongating and advancing are performed simultaneously or sequentially.

13. A device for separating soft tissues for cannula access to a treatment site within a patient body, the device comprising:
a catheter body having a proximal end and a distal end;
an expansible tissue distractor disposed on the distal end of the catheter; and
a mechanical actuation mechanism coupled to the tissue distractor for elongating the tissue distractor.

14. The device of claim 13, wherein the tissue distractor comprises a balloon.

15. The device of claim 14, further comprising an inflation assembly in fluid communication with the balloon so as to inflate the balloon, prior to elongation, to an expanded diameter.

16. The device of claim 15, wherein the expanded balloon diameter is in a range from about 5 mm to about 50 mm.

17. The device of claim 16, wherein the expanded balloon diameter is larger than a diameter of the cannula.

18. The device of claim 15, wherein the expanded balloon has a volume in a range from about 3 cc to about 30 cc.

19. The method of claim 18, wherein the balloon volume remains substantially constant prior to and subsequent to elongation.

20. The device of claim 14, wherein the mechanical actuation mechanism comprises an inner shaft of the catheter that is distally moveable relative to an outer shaft of the catheter.

21. The device of claim 20, further comprising a plunger coupled to a proximal end of the inner shaft.

22. The device of claim 14, wherein the mechanical actuation mechanism comprises an outer shaft of the catheter that is proximally moveable relative to an inner shaft of the catheter.

23. The device of claim 20 or 22, wherein a proximal end of the balloon is coupled to a distal end of the outer shaft and a distal end of the balloon is coupled to a distal end of the inner shaft.

24. The device of claim 20 or 22, wherein the balloon has a variable length in a range from about 40 mm to about 150 mm.

25. The device of claim 24, wherein the balloon is elongated in an axial direction.

26. The device of claim 25, wherein the balloon has an elongated length in a range from about 60 mm to about 180 mm.

27. The device of claim 25, wherein an outer diameter of the elongated balloon is reduced by a range from about 0.1 mm to about 10 mm.

28. The device of claim 27, wherein the elongated balloon diameter is smaller than a diameter of the cannula.

29. A system for separating spinal tissues for cannula access to a spine within a patient body, the system comprising:
a catheter body having a balloon tissue distractor disposed on a distal end thereof configured to separate spinal tissues near the spine and a mechanically actuatable elongation mechanism coupled to the balloon tissue distractor; and
a cannula disposed over the catheter body.
